(19) United States
(12) Patent Application Publication
(10) Pub. No.: US 2025/0295936 A1
(43) Pub. Date: Sep. 25, 2025

(54) AI-ENHANCED NON-IMAGING TUS SYSTEMS

(71) Applicant: Sanmai Technologies, PBC, Sunnyvale, CA (US)

(72) Inventors: Christopher DAFT, Tucson, AZ (US); Sharena RICE, Sunnyvale, CA (US); Sarp SATIR, Oakland, CA (US)

(21) Appl. No.: 19/033,083

(22) Filed: Jan. 21, 2025

Related U.S. Application Data
(60) Provisional application No. 63/567,382, filed on Mar. 19, 2024.

(51) Int. Cl. A61N 7/00; G06T 7/00; G06T 7/11; G06T 11/00

(52) U.S. Cl. CPC A61N 7/00; G06T 7/0012; G06T 7/11; G06T 11/001; A61N 2007/0026; A61N 2007/0095; G06T 2207/10088; G06T 2207/20081; G06T 2207/30016; G06T 2210/41

(57) ABSTRACT

Transcranial ultrasound systems (TUS) and methods use domain-specific large vision models (DSLVM) artificial intelligence systems to improve the efficacy of non-imaging probes. A positioning DSLVM assists an operator in improving the placement of the probe on a patient's head. The positioning DSLVM uses a pre-procedure MRI of the patient's head, target dose plan, target anatomy, and the probe's position information on the scalp, and it outputs the control parameters for the probe's beamformer. A segmenting DSLVM helps an operator with the optimal initial placement of the non-imaging probe by highlighting anatomical structures in color.

### CROSS-REFERENCE TO RELATED APPLICATIONS

This application claims the priority benefit of U.S. Provisional Application No. 63/567,382, filed Mar. 19, 2024, and entitled, “AI-ENHANCED NON-IMAGING TUS SYSTEMS”. The subject matter of this related application is hereby incorporated herein by reference.

### TECHNICAL FIELD

This invention relates to transcranial ultrasound systems (TUS) that employ domain specific large vision models (DSLVM) to assist in the reliable therapeutic ultrasound (US) delivery to the targeted brain anatomy.

### BACKGROUND

TUS systems help treat several types of mental illness, but current systems do not guarantee that the ultrasound stimulation accurately reaches the anatomical targets. FIGS. 1A-1E are prior art illustrations highlighting a human head's relevant anatomical structures (or tissues). FIG. 1A shows the human head 101 and highlights skull bone 110. FIG. 1B highlights cerebrospinal fluid 120. FIG. 1C highlights nervous tissue 130. FIG. 1D and FIG. 1E highlight air 140 and muscle 150, respectively. These anatomical structures complicate targeting ultrasonic stimulation. These structures have different acoustic properties, which refract, reflect, and diffract the ultrasound field. Critically, air cavities 140 must be avoided, which inhibits US propagation. As partial ultrasound delivery to the target reduces efficacy and may create unwanted side effects, the operator must adjust the system for each structure type. System operators must choose optimal probe locations and use their skills to avoid sub-optimal US paths. The operating complexity of a TUS can be reduced by identifying structures within the head to assist the less-skilled operator. The system must assist the operators by identifying sub-optimal paths such as air cavities 140 and thick or curved portions of the skull bone 110. Reducing the overall cost and complexity is necessary to make the systems widely usable.

### BRIEF DESCRIPTION OF THE DRAWINGS

FIGS. 1A-1E are prior art illustrations highlighting the relevant anatomical structures of a human head.

FIG. 2 shows an AI-enhanced non-imaging TUS system 200.

FIG. 3 is a functional block diagram illustrating training for positioning LVM 220 using a set of MRIs and probe 210 locations.

FIG. 4 is a functional block diagram illustrating training for segmenting LVM 240 using a set of MRIs.

FIG. 5 is an exemplary method 500 that uses positioning LVM 220 to assist an operator in positioning a simple-probe TUS system 200.

FIG. 6 is an exemplary method 600 that uses segmenting LVM 240 and positioning LVM 220 to assist an operator in positioning a simple-probe TUS system 200.

### DETAILED DESCRIPTION

Transcranial ultrasound systems (TUS) and methods use domain-specific large vision models (DSLVM) artificial intelligence systems to improve the efficacy of non-imaging probes. A positioning DSLVM assists an operator in improving the placement of the probe on a patient's head. The positioning DSLVM uses a pre-procedure MRI of the patient's head, target dose plan, target anatomy, and the probe's position information on the scalp, and it outputs the control parameters for the probe's beamformer. A segmenting DSLVM helps an operator with the optimal initial placement of the non-imaging probe by highlighting anatomical structures in color.

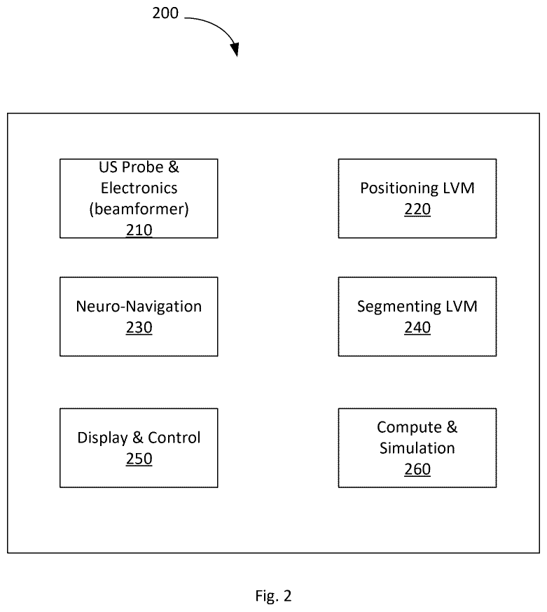

FIG. 2 shows an AI-enhanced non-imaging TUS system 200. This application discloses methods and systems for reliably using simple, low-frequency probe (probe arrays or probes) enhanced with artificial intelligence systems to deliver ultrasound to targeted brain anatomy. Examples of these probes are:

An annular array with several rings—as few as four rings (termed Type 1 probes.)

A low-element-count non-imaging array, such as a 12×12- or 16×16-element array (termed Type 2 probes). This kind of array can steer and focus but may still lack imaging capabilities to guide the stimulation because its center frequency is too low to provide adequate resolution.

In this application, Type 1 annular arrays and Type 2 low-element-count non-imaging arrays collectively are referred to as “simple probes” to distinguish them from probes capable of imaging inside the skull to provide stimulation guidance. Simple probes may be used for tFUS (transcranial focused ultrasound) or TUS treatment if some means other than ultrasound can guide the stimulation beam. Referring to FIG. 2, System 200 includes simple probes 210. Associated with probe 210 are the electronics needed to control the US probe, such as beamformer, etc. Probe 210 is placed on the user, subject, or patient's head. User, subject, and patient are used interchangeably and have the same meaning in this application. Neuro-Navigation 230 computes the position and orientation (pose) of probe 210 on the user's head. The neuro-navigation data is provided to Compute & Simulation block 260. Neuro-navigation 230 reduces the cost of System 200 as it does not need a display and sophisticated software. Neuro-navigation 230 reduces the cost and complexity by using only two infrared cameras and infrared LED sources. The embedded computation can be done with small, power-efficient, and highly cost-effective hardware like Rock5 or Raspberry PI.

Display & Control 250 block allows the operator to interact with System 200. Block 250 can be an App on a smartphone or tablet interfacing with System 200 using a wireless protocol such as Bluetooth, WiFi, etc. In the preferred embodiment, Block 250 is operated on a dedicated device with a touchscreen or other means of controlling it, such as a mouse and a keyboard. The dedicated device only interfaces with System 200 and no other device. The operator uses Block 250 to specify the target location(s) and dose distribution specification (Ultrasound properties-frequency, intensity, time, repeat frequency). An operator may use a textual or graphical input to provide the target location(s) and dose distribution to System 200. System 200 provides instructions or other messages to the operator using Block 250.

Compute & Simulation block 260 generates an ultrasound field based on the probe's location & orientation and a pre-procedure MRI (Magnetic Resonance Imaging) of the subject's head.

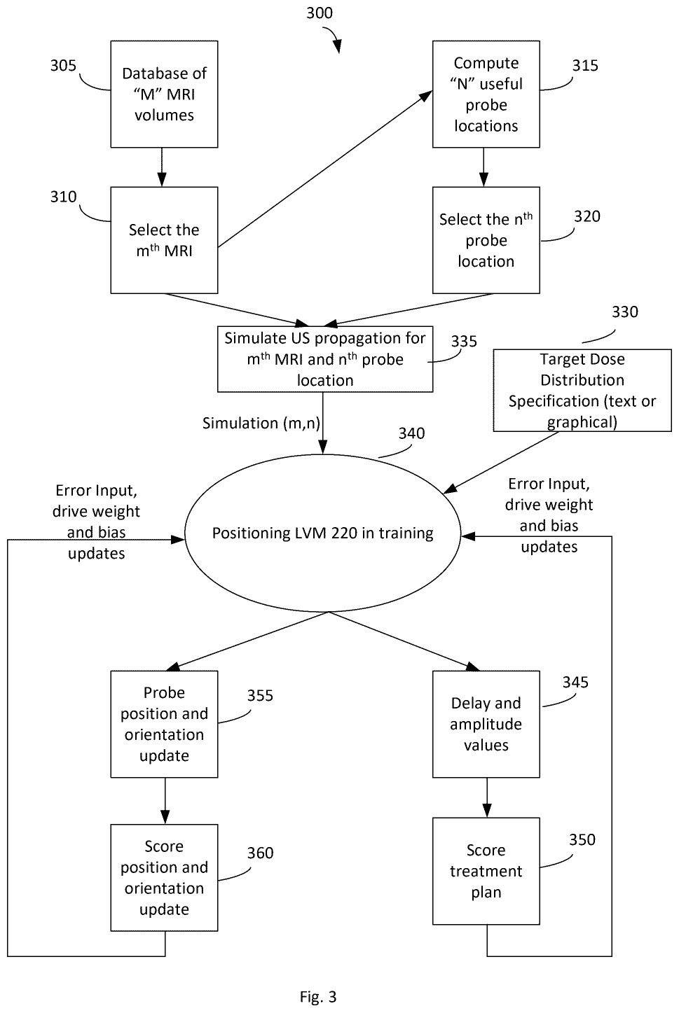

System 200 uses artificial intelligence (AI) systems to enhance the efficacy of US treatment. It includes a positioning LVM 220 (large vision models or domain-specific large vision models, LVM and DSLVM are here used interchangeably) to assist the operator in determining the optimal location and orientation of probe 210. The inputs provided to the positioning DSLVM 220 are a pre-procedure MRI of the patient's head, the target dose specification, and a dynamic prompt of the ultrasound field. DSLVM 220 has learned how (described in FIG. 3) to take the prediction of the ultrasound field distribution with the current probe 210 position & orientation, the specified dose distribution, and the overall anatomy provided by the MRI and compute an improvement of the probe position & orientation. DSLVM used in System 200 can be attention-based (e.g., a transformer neural network), or of convolutional type (CNN), or a multi-layer perceptron (MLP). DSLVM 220 improves the initial approximate probe position by providing a recommended update to the probe location and orientation, which improves the stimulation field based on whether the probe location and orientation are targeting the target anatomy. The outputs of DSLVM 220 include a set of parameters to program a stimulation beamformer. LVM 220 outputs the data to program the beamformer based on the clinician's specification of the desired ultrasonic dose distribution within the target tissue. This can be achieved by programming the beamformer for a particular treatment plan, and/or making the treatment comprise multiple beams. The stimulation beamformer is configured to steer an ultrasonic stimulation beam to a region defined in the outline of the target tissue.

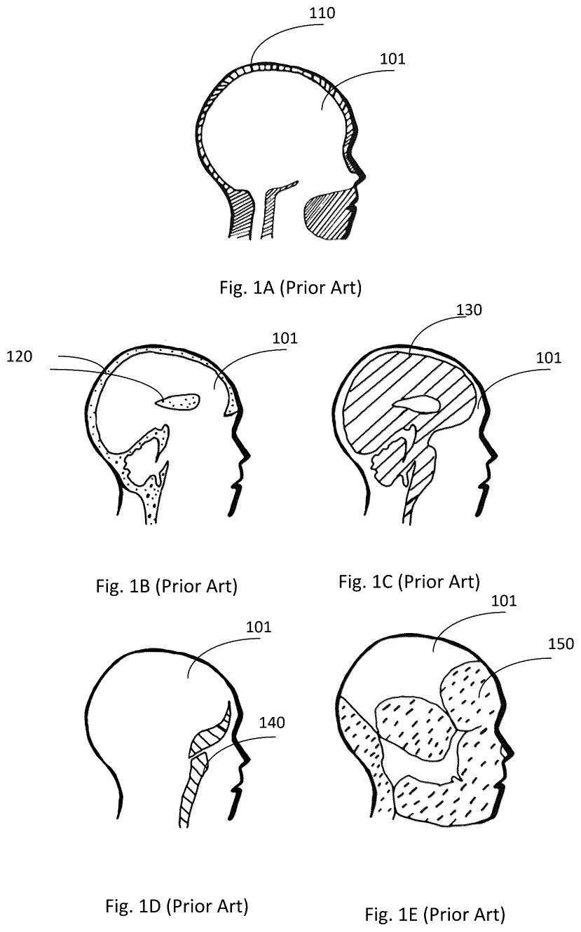

System 200 employs a Segmenting LVM 240 to allow the operator to gain confidence that their initial probe positioning is near optimal. The pre-procedure MRI of the subject's head is segmented and displayed as a rendered volume on display 250, or as a number of slices that the operator can scroll through. Different cranial anatomical structures and the clinician-specified treatment plan are highlighted in color on the grayscale structural MRI. The operator can assess whether a particular probe position will be problematic due to bone structures 110, air pockets 140, or other anatomical features like those shown in FIG. 1.

Compute & Simulation block 260 simulates the ultrasound field using the patient's pre-procedure MRI and probe 210's position and orientation. This simulation is a pre-processing step used assist LVM 220. In an embodiment, only the probe 210's position and orientation, a static pre-procedure MRI of the subject's head, and the desired dose distribution are provided as inputs to LVM 220. The representation of the acoustic field is realized within the LVM.

### Positioning LVM Training

FIG. 3 is a functional block diagram illustrating training for positioning LVM 220 using a set of MRIs and probe 210 locations. Probe positioning DSLVM 220 is trained with synthetic data to estimate an update to the probe's position and the beamforming parameters appropriate for the new position and orientation. The synthetic data includes an MRI database 305 and a simulation of an ultrasound field 335 produced by probe 210 at a certain location and orientation (315) on the subject's head.

With the same MRI 310, simulations of the ultrasound field are produced with the probe at many positions (labeled 315 & 320 in FIG. 3) on the head. A library (or database) of M MRI scans 305 is used for training, and N probe positions 315 are simulated for each MRI scan, resulting in MN pairs of items in the training set.

The DSLVM in training 340 is also provided with information about the target 330, either in textual form (e.g., “left amygdala”) or graphically. The operator can produce graphical indications of the target outline by annotating the MRI scan using DICOM display software. The software can deliver a set of points to the DSLVM. The operator may supply more data than simply an outline of the target anatomical structure. A desired dose distribution may also be provided in cases where a varying amount of stimulation throughout the target structure has more clinical efficacy.

The DSLVM internally learns the coordinate transformation between the MRI and the ultrasound simulation during this training. When presented with an MRI and an ultrasound scan from a particular probe position, it proposes a new location and orientation 355 for the probe and beamforming delays for the probe elements.

The estimates 345 and 355 go into a scoring system 350 and 360 which include a forward ultrasound simulation using the proposal and evaluates the closeness of the simulation to the clinician's specification. The scoring is based on the distribution of the acoustic dose within the target anatomical structure and the amount of sound energy deposited outside the target. The DSLVM is penalized for a probe position and delay values where there is substantial stray ultrasound outside the target structure(s). It is rewarded for choices that produce a dose distribution within the target that matches the treatment plan. The center of the beam after the update should be near the spatial center of gravity of the target.

For Type 1 probes (Annular arrays), the DSLVM outputs delays (345) for each annular ring to create a peak within the treatment structure. For Type 2, a set of delay matrices (345) may be produced. The aim is to fill the target with a pre-specified dose distribution by steering and focusing a plurality of ultrasound beams. The scoring (350) compares the realized dose to the plan specified by the clinician.

The scores are fed back to the DSLVM as error metrics to minimize. After ingesting a large training set, the model learns an internal representation of the design space and can then provide an update on the probe position and beamforming delays, which works well for a wide patient population.

### Segmenting LVM Training

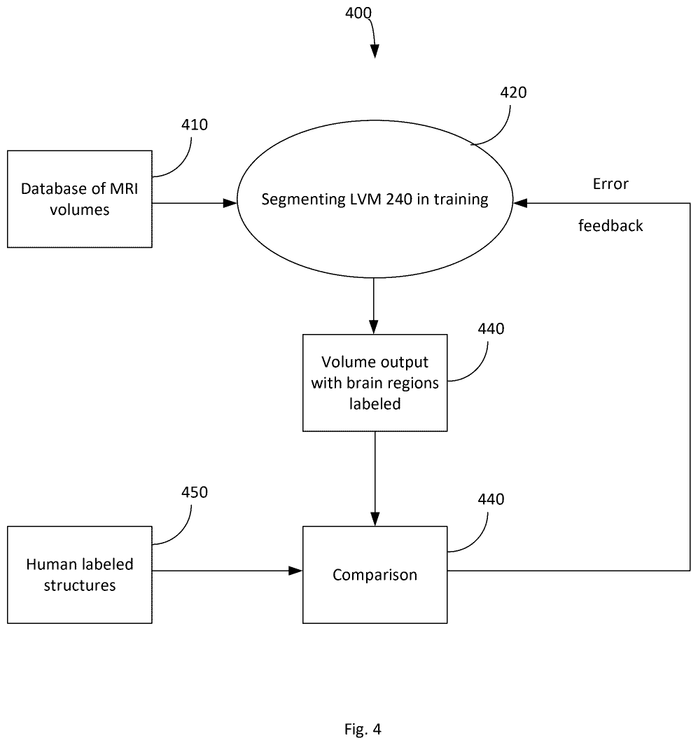

FIG. 4 is a functional block diagram illustrating training for segmenting LVM 240 using a set of MRIs. As illustrated in FIG. 4, a standard supervised training procedure is used in which LVM 240 learns the types of anatomical structures (shown in FIG. 1). A database 410 of MRIs is presented to the Segmenting LVM 240 in training 420. Segmenting LVM 240 outputs volume outputs with brain regions 440. The output is compared (440) to human-labeled structures 450 to create error feedback into the segmenting LVM 240 in training 420.

### Positioning Method Using Positioning LVM

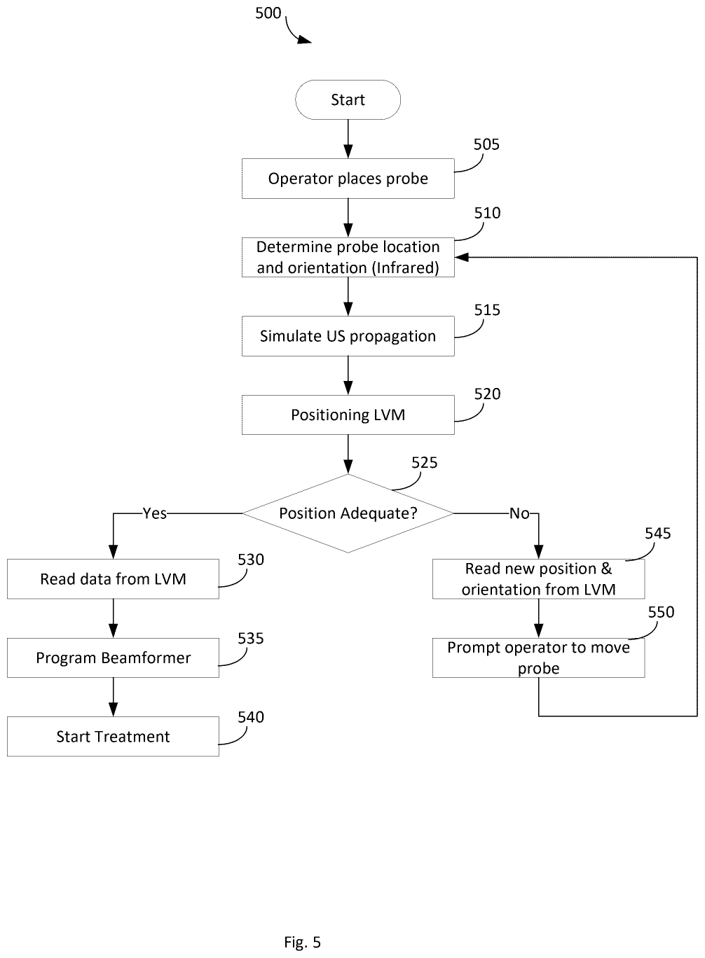

FIG. 5 is an exemplary method 500 that uses positioning LVM 220 to assist an operator in positioning a simple-probe TUS system 200. Method 500 uses a pre-procedure structural MRI of the patient's head and probe 210's position and orientation on the patient's head from the Neuro-navigation 230. LVM 220 in Method 500 produces a recommendation to the operator on how to move probe 210 to improve the treatment. Method 500 or process can be accomplished without the operator's significant anatomical knowledge. The operator needs to understand where a reasonable starting position on the head is for the specific treatment and attach the probe near that point. All adjustments from the initial positioning are automatic. Method 500 also provides the parameters for programming the beamformer of probe 210. The treatment may consist of multiple beams.

In FIG. 5, an operator places probe 210 on the subject's head in Operation 505.

In Operation 510, Neuro-navigation 230 determines probe 210's position (location on the patient's head) and orientation. In an embodiment, Neuro-navigation 230 uses an infrared-based system consisting of two infrared cameras and infrared LED sources. The probe (on a headset) includes features that reflect infrared light transmitted by the LEDs. Two infrared cameras capture the light from infrared LEDs. This information is used to compute probe 210's position and orientation.

In Operation 515, an ultrasound simulation is performed. The simulation uses the MRI and probe's positions to compute the field the probe would produce if the stimulation were turned on at the current position. The field is presented to the DSLVM as a visual prompt. In an embodiment, Operation 515 is optional. Based on the computing capabilities of DSLVM, the acoustic field can be realized within LVM 220.

In Operation 520, with the static input of the MRI, the target dose specification, and the dynamic prompt of the ultrasound field, DSLVM 220 provides an update to the probe 210's position. It has learned how to take the prediction of where the ultrasound will go with the current probe position, the specified dose distribution, and the overall anatomy as provided by the MRI and compute an improvement to the probe 210's position and orientation.

In Operation 525, Method 500 determines whether the current probe 210's position is adequate by comparing the position update proposed by the LVM to the current position. If the difference between the probe positions is smaller than an empirically determined threshold, the following operation is Operation 530. If not, the following operation is Operation 545.

Data from LVM 220 is read in Operation 530. These data are used to program the electronics (beamformer) of probe 210 in Operation 535. For Type 1 probes (Annular arrays), the DSLVM outputs delays for each annular ring to create a peak within the treatment structure. For Type 2, a set of delay matrices may be produced. The aim is to fill the target with a pre-specified dose distribution by steering and focusing a plurality of ultrasound beams. The LVM outputs the data to program the beamformer based on the clinician's specification of the desired ultrasonic dose distribution within the target. This is achieved by programming the beamformer and making the treatment comprise multiple beams.

The patient's treatment starts in Operation 540. If, during the treatment, the operator determines that probe 210's position or orientation needs an update, Method 500 moves to Operation 505.

In Operation 545, the new position and orientation of probe 210 are read from LVM 220.

In Operation 550, the operator is prompted to move probe 210. The prompt can be visual or auditory. As the probe 210 approaches the target location, visual or auditory clues are provided to the operator. The prompting can be achieved by superimposing arrows on an image of the subject and displayed on Display 250. As the proposed new location approaches, the arrows change direction and confirm that the operator has found the correct location. While the operator moves the probe, Method 500 continuously updates its location from the Neuro-navigation system and compares it with the LVM proposed update.

### Requirements for LVM Inputs

LVM 220 maps a static MRI, a dose distribution, and a simulation to predict the ultrasound field into an update to the current probe position and orientation. The simulation output makes the obstacles to treatment shown in FIG. 1 very clear to the model.

However, the simulation step is a type of pre-processing, much like Principal Components Analysis, which is used to reduce dimensionality in many machine learning applications. With enough LVM power, Operation 515 can be removed from Method 500. Instead, the LVM is provided with only the probe position and orientation, the static MRI, and the desired dose distribution. The representation of the acoustic field is then realized only in the LVM's latent space.

### Enhanced Positioning Method Using Segmenting LVM

A segmented MRI can help the operator gain confidence that their initial probe positioning is near optimal. Here, the pre-procedure MRI is segmented and displayed as a volume, perhaps with the different anatomical structure types highlighted in color on the otherwise greyscale structural MRI. The operator can assess whether a certain probe position will be problematic because of thick bone 110, air pockets 140, or other anatomical issues. The initial probe placement is made in the light of the segmented volume scan.

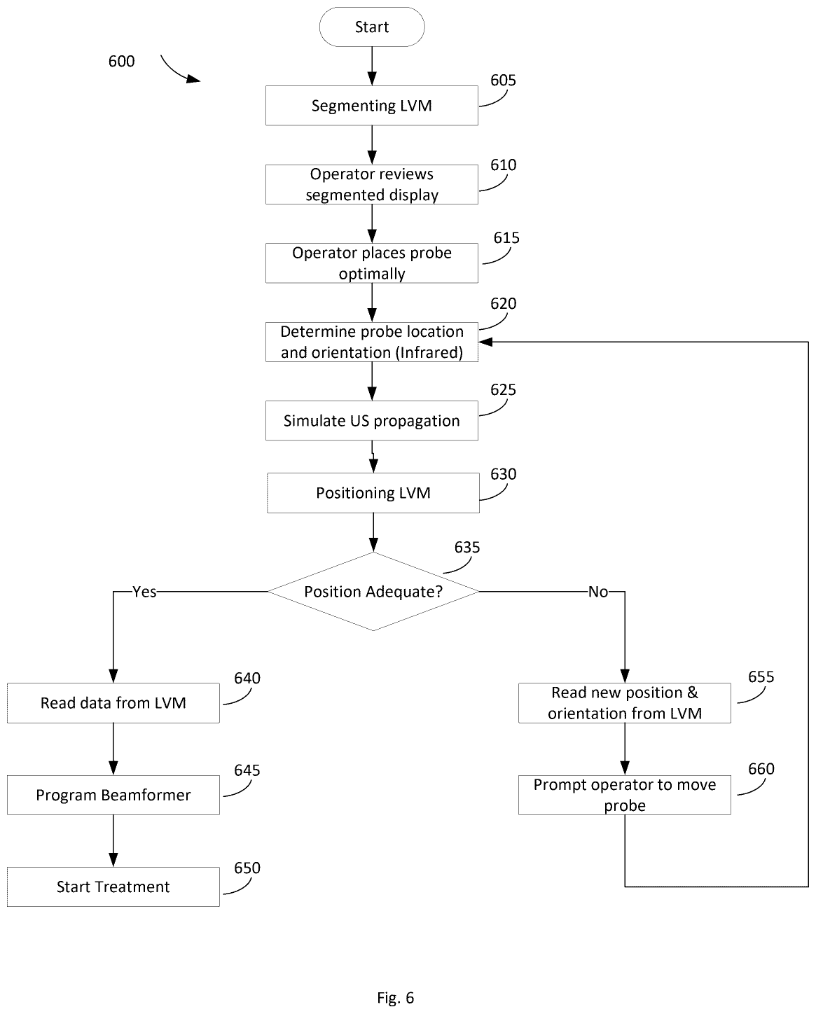

FIG. 6 is an exemplary method 600 that uses segmenting LVM 240 and positioning LVM 220 to assist an operator in positioning a simple-probe TUS system 200. Method 600 invokes segmenting LVM 240, provided with a pre-procedure structural MRI of the patient's head. LVM 240 displays a segmented display on display 250. The pre-procedure MRI is segmented and displayed as a volume, with the different anatomical structure types and the stimulation target highlighted in color on the otherwise greyscale structural MRI. Method 600 also provides the parameters for programming the beamformer of probe 210. The treatment may consist of multiple beams.

In Operation 610, the operator reviews the segmented display and determines the optimal placement for probe 210.

In Operation 615, the operator optimally places probe 210.

In Operation 620, Neuro-navigation 230 determines probe 210's position (location on the patient's head) and orientation. In an embodiment, Neuro-navigation 230 uses an infrared-based system consisting of two infrared cameras and an infrared LED source. The probe (on a headset) includes features that reflect infrared light transmitted by the LEDs. Two infrared cameras capture the light from infrared LEDs. This information is used to compute probe 210's position and orientation.

In Operation 625, an ultrasound simulation is performed. The simulation uses the MRI and probe's positions to compute the field the probe would produce if the stimulation were turned on at the current position. The field is presented to the DSLVM as a visual prompt. In an embodiment, Operation 625 is optional. Based on the computing capabilities of DSLVM, the acoustic field can be realized within LVM 220.

In Operation 630, with the static input of the MRI, the target dose specification, and the dynamic prompt of the ultrasound field, DSLVM 220 provides an update to the probe 210's position. It has learned how to take the prediction of where the ultrasound will go with the current probe position, the specified dose distribution, and the overall anatomy as provided by the MRI and compute an improvement to the probe 210's position and orientation.

In Operation 625, Method 600 determines whether the current position of probe 210 is adequate by comparing the position update proposed by the LVM to the current position. If the difference between the probe positions is smaller than an empirically determined threshold, the following operation is Operation 640. If not, the following operation is Operation 655.

Data from LVM 220 is read in Operation 640. This data is used to program the electronics (beamformer) of probe 210 in Operation 645. For Type 1 probes (Annular arrays), the DSLVM outputs delays (345) for each annular ring to create a peak within the treatment structure. For Type 2, a set of delay matrices (345) may be produced. The aim is to fill the target with a pre-specified dose distribution by steering and focusing a plurality of ultrasound beams. The LVM outputs the data or parameters to program the beamformer based on the clinician's specification of the desired ultrasonic dose distribution within the target. This is achieved by programming the beamformer and making the treatment comprise multiple beams.

The patient's treatment starts in Operation 650. If, during the treatment, the operator determines that probe 210's position or orientation needs an update, Method 600 moves to Operation 615.

In Operation 655, the new position and orientation of probe 210 are read from LVM 220.

In Operation 660, the operator is prompted to move probe 210. The prompt can be visual or auditory. The prompting can be achieved by superimposing arrows on an image of the subject and displayed on Display 250. As the proposed new location approaches, the arrows change direction and confirm that the operator has found the correct location. While the operator moves the probe, Method 600 continuously updates its location from the Neuro-navigation system and compares it with the LVM proposed update.

Operations 655 and 660 in Method 600 help eliminate any operator errors. Operator errors may occur due to incorrect interpretation of segmented display in Operation 610 or incorrect placement in Operation 615.
1. In some embodiments, a non-imaging system for ultrasonically treating a target anatomy comprises an ultrasonic probe, a neuro-navigation subsystem that determines a position of the ultrasonic probe, a control subsystem in communication with an input device, wherein the control subsystem receives a selected treatment associated with the ultrasonic probe, a compute and simulation subsystem configured to generate an ultrasonic field emitted by the ultrasonic probe based upon the selected treatment, the position, and patient data, and a large vision model (DSLVM) executed by at least one processor that generates an adjusted location associated with the ultrasonic probe based upon ultrasonic field, the selected treatment and the position of the ultrasonic probe, wherein the control subsystem transmits the adjusted location to the input device.
2. The system of clause 1, wherein the DSLVM generates an adjusted location and orientation to the position of the ultrasonic probe, wherein the adjusted location and orientation are associated with a target anatomy based on whether the probe location and orientation are targeting the target anatomy.
3. The system of clauses 1 or 2, wherein the DSLVM further generates a set of parameters to program a stimulation beamformer associated with the ultrasonic probe.
4. The system of any of clauses 1-3 wherein the stimulation beamformer is configured to steer and focus an ultrasonic stimulation beam emitted by the ultrasonic probe to a region defined by the selected treatment.
5. The system of any of clauses 1-4, wherein the selected treatment is provided to the DSLVM by a text prompt input by an operator.
6. The system of any of clauses 1-5 wherein the DSLVM generates the adjusted location based upon a target structure specification received from specification that is graphically input.
7. The system of any of clauses 1-5 wherein the operator's selection of the target structure(s) is aided by a segmented MRI showing various anatomical structure types.
8. The system of any of clauses 1-7, wherein the specified treatment comprises a desired ultrasonic dose distribution within a target that is achieved by programming a beamformer associated with the ultrasonic probe, wherein the specified treatment comprises multiple beams.
9. In some embodiments, a method comprises obtaining a structural magnetic resonance image (MRI) associated with a patient, providing the MRI to an input device, receiving, from the input device, a selected treatment associated with an ultrasonic probe, and generating, based upon the selected treatment, a probe location, and the MRI, a recommended probe location and a dose distribution specification associated with ultrasonic field emitted by the ultrasonic probe.
10. The method of clause 9, further comprising simulating an ultrasound propagation based on the MRI, the probe location and the selected treatment.
11. The method of clauses 9 or 10, further comprising determining the probe location based upon an infrared-based neuro-navigation system.
12. The method of any of clauses 9-11, further comprising uses generating an adjusted probe location using a large vision model (LVM) based on the probe location, the MRI, and the dose distribution specification.
13. The method of any of clauses 9-12, wherein the LVM outputs parameters to program a beamformer associated with the ultrasonic probe.
14. The method of any of clauses 9-13, where the selected treatment comprises a plurality of beams emitted by the ultrasonic probe.
15. The method of any of clauses 9-14, wherein the LVM generates a recommendation to move the probe to improve the selected treatment.
16. The method of any of clauses 9-15, where the operator is provided with visual and auditory prompts to move the probe.
17. In some embodiments, a method comprises obtaining a structural magnetic resonance image (MRI) associated with a patient, segmenting the MRI using a large vision model (DSLVM), colorizing a stimulation target using the DSLVM, providing the segmented and colorized MRI to an input device, receiving, from the input device, a selected treatment associated with an ultrasonic probe, and generating, based upon the selected treatment, a probe location, and the segmented and colorized MRI, a recommended probe location and a dose distribution specification associated with ultrasonic field emitted by the ultrasonic probe.
18. The method of clause 17, further comprising colorizing a plurality of anatomical structure types within the MRI using the DSLVM.
19. In some embodiments, a method comprises obtaining a database storing a plurality of magnetic resonance images (MRI) associated with a plurality of patients, for at least a subset of the plurality of MRI from the database generating a simulation of an ultrasound field produced by a probe applied to at least a subset of the plurality of MRI from the database, and receiving an identification of a target region associated with the selected MRI from the database, and training a large vision model (DSLVM) based on the simulation of the ultrasound field and the identification of the target region.
20. The method of clause 19, wherein training the DSLVM further comprises generating a location and orientation of the probe for a respective one of the MRI's, scoring the generated location and orientation based upon a closeness of the generated location and orientation to the target region associated with the respective one of the MRI's, and training the DSLVM based upon the score.
21. The method of clauses 19 or 20, wherein training the DSLVM further comprises generating a delay associated with the probe for a respective one of the MRI's, scoring the generated location and orientation based upon a realized dose of treatment associated with the probe to a plan associated with the respective one of the MRI's, and training the DSLVM based upon the score.
22. In some embodiments, a method comprises obtaining a database storing a plurality of magnetic resonance images (MRI) associated with a plurality of patients, wherein each of the MRI's is associated with an identified anatomical structure, and training a segmenting large vision model based on the database, based on an identified brain region output by the segmenting large vision model and a respective human-label structure corresponding to the identified anatomical structure.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, for example, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A non-imaging system for ultrasonically treating a target anatomy comprising:
an ultrasonic probe;
a neuro-navigation subsystem that determines a position of the ultrasonic probe;
a control subsystem in communication with an input device, wherein the control subsystem receives a selected treatment associated with the ultrasonic probe;
a compute and simulation subsystem configured to generate an ultrasonic field emitted by the ultrasonic probe based upon the selected treatment, the position, and patient data; and
a large vision model (DSLVM) executed by at least one processor that generates an adjusted location associated with the ultrasonic probe based upon ultrasonic field, the selected treatment and the position of the ultrasonic probe, wherein the control subsystem transmits the adjusted location to the input device.

2. The system of claim 1, wherein the DSLVM generates an adjusted location and orientation to the position of the ultrasonic probe, wherein the adjusted location and orientation are associated with a target anatomy based on whether the probe location and orientation are targeting the target anatomy.

3. The system of claim 1, wherein the DSLVM further generates a set of parameters to program a stimulation beamformer associated with the ultrasonic probe.

4. The system of claim 3 wherein the stimulation beamformer is configured to steer and focus an ultrasonic stimulation beam emitted by the ultrasonic probe to a region defined by the selected treatment.

5. The system of claim 1, wherein the selected treatment is provided to the DSLVM by a text prompt input by an operator.

6. The system of claim 1 wherein the DSLVM generates the adjusted location based upon a target structure specification received from specification that is graphically input.

7. The system of claim 1 wherein the operator's selection of the target structure(s) is aided by a segmented MRI showing various anatomical structure types.

8. The system of claim 3, wherein the specified treatment comprises a desired ultrasonic dose distribution within a target that is achieved by programming a beamformer associated with the ultrasonic probe, wherein the specified treatment comprises multiple beams.

9. A method comprising:
obtaining a structural magnetic resonance image (MRI) associated with a patient;
providing the MRI to an input device;
receiving, from the input device, a selected treatment associated with an ultrasonic probe; and
generating, based upon the selected treatment, a probe location, and the MRI, a recommended probe location and a dose distribution specification associated with ultrasonic field emitted by the ultrasonic probe.

10. The method of claim 9, further comprising simulating an ultrasound propagation based on the MRI, the probe location and the selected treatment.

11. The method of claim 9, further comprising determining the probe location based upon an infrared-based neuro-navigation system.

12. The method of claim 9, further comprising uses generating an adjusted probe location using a large vision model (LVM) based on the probe location, the MRI, and the dose distribution specification.

13. The method of claim 12, wherein the LVM outputs parameters to program a beamformer associated with the ultrasonic probe.

14. The method of claim 9, where the selected treatment comprises a plurality of beams emitted by the ultrasonic probe.

15. The method of claim 12, wherein the LVM generates a recommendation to move the probe to improve the selected treatment.

16. The method of claim 15, where the operator is provided with visual and auditory prompts to move the probe.

17. A method comprising:
obtaining a structural magnetic resonance image (MRI) associated with a patient;
segmenting the MRI using a large vision model (DSLVM);
colorizing a stimulation target using the DSLVM;
providing the segmented and colorized MRI to an input device;
receiving, from the input device, a selected treatment associated with an ultrasonic probe; and
generating, based upon the selected treatment, a probe location, and the segmented and colorized MRI, a recommended probe location and a dose distribution specification associated with ultrasonic field emitted by the ultrasonic probe.

18. The method of claim 17, further comprising colorizing a plurality of anatomical structure types within the MRI using the DSLVM.

19. A method comprising:
obtaining a database storing a plurality of magnetic resonance images (MRI) associated with a plurality of patients;
for at least a subset of the plurality of MRI from the database:
generating a simulation of an ultrasound field produced by a probe applied to at least a subset of the plurality of MRI from the database; and
receiving an identification of a target region associated with the selected MRI from the database; and
training a large vision model (DSLVM) based on the simulation of the ultrasound field and the identification of the target region.

20. The method of claim 19, wherein training the DSLVM further comprises:
generating a location and orientation of the probe for a respective one of the MRI's;
scoring the generated location and orientation based upon a closeness of the generated location and orientation to the target region associated with the respective one of the MRI's; and
training the DSLVM based upon the score.

21. The method of claim 19, wherein training the DSLVM further comprises:
generating a delay associated with the probe for a respective one of the MRI's;
scoring the generated location and orientation based upon a realized dose of treatment associated with the probe to a plan associated with the respective one of the MRI's; and
training the DSLVM based upon the score.

22. A method comprising:
obtaining a database storing a plurality of magnetic resonance images (MRI) associated with a plurality of patients, wherein each of the MRI's is associated with an identified anatomical structure; and
training a segmenting large vision model based on the database, based on an identified brain region output by the segmenting large vision model and a respective human-label structure corresponding to the identified anatomical structure.
